(19) United States
(12) Patent Application Publication
(10) Pub. No.: US 2011/0046202 A1
(43) Pub. Date: Feb. 24, 2011

(54) METHOD FOR TESTING A SUBJECT THOUGHT TO HAVE OR TO BE PREDISPOSED TO ASTHMA

(76) Inventors: Miriam Fleur Moffat, London (GB); William Osmond Charles Cookson, London (GB); Ivo Glynne Gut, Evry (FR); Gregory Mark Lathrop, Evry (FR); Michael Kabesch, Munich (DE); Martin Farral, Oxford (GB); Goncalo Rocha Abecasis, Ann Arbor, MI (US); Liming Liang, Ann Arbor, MI (US)

Correspondence Address: FOLEY AND LARDNER LLP;SUITE 500, 3000 K STREET NW, WASHINGTON, DC, 20007, US

(21) Appl. No.: 12/665,602

(22) PCT Filed: Jun. 19, 2008
(86) PCT No.: PCT/EP08/57818

§ 371 Date: Oct. 29, 2010

(30) Foreign Application Priority Data
Jun. 20, 2007 (EP) 07301135.5

(51) Int. Cl. A61K 31/713; C12Q 1/68; C40B 30/04; A61P 11/06; A61P 37/08

(52) U.S. Cl. 514/44 A; 435/6; 506/9

(57) ABSTRACT

The present invention concerns a method of testing a subject thought to have or be predisposed to having asthma, allergy, atopic disease or atopic sensitization, which comprises the step of analyzing a biological sample from said subject for (i) detecting the presence of a mutation associated with the over-expression of the ORMDL3 gene, and/or (ii) analyzing the expression of the ORMDL3 gene; a use, for treating and/or preventing asthma, allergy, atopic disease or atopic sensitization in a subject, of a compound which specifically inhibits the expression of the ORMDL3 gene; and an in vitro method of selecting a compound, which can be useful for treating asthma, allergy, atopic disease or atopic sensitization, characterized in that said method comprises the steps of: (a) obtaining a cell expressing the ORMDL3 gene, (b) contacting said cell with at least one compound, (c) comparing the expression of the ORMDL3 gene in the cell between the steps a) and b), and (d) selecting the compound, which induces a lower level of expression of the ORMDL3 gene in the cell contacted to that compound.

This invention claims the priority of the European patent application EP07301135.5, filed on Jun. 20, 2007, which is incorporated herein by reference.

### FIELD OF THE INVENTION

The present invention relates to asthma, allergies, atopic diseases or atopic sensitization, and more precisely to a method of testing a subject thought to have or to be predisposed to asthma, allergies, atopic diseases or atopic sensitization.

### BACKGROUND OF THE INVENTION

Asthma is a chronic inflammatory disorder and, in genetically-susceptible individuals, this inflammation leads to increased airway responsiveness to a variety of stimuli, and recurrent airway obstruction. It is the most common chronic disease of childhood and the most common reason for pediatric hospital admission. Although it is clear that both environmental and genetic influences are important in the development of asthma, the pathogenesis of this disease remains unclear.

Delineating the genes which contribute to the development of asthma, and dissecting the mechanisms by which these genes alter the host response to environmental challenge (antigen, viral, etc.) are key steps to furthering our knowledge of the pathogenesis of asthma.

There are essentially two primary approaches to gene discovery that are being used in the context of asthma and allergy: those using a candidate gene evaluation and those using a genome-wide linkage and association scan.

The motivation of the candidate gene study is that the candidate might be in a pathway involved in disease and might have a functional polymorphism that can be tested for association with asthma in a population. A typical example of such a study is given in WO03/073990 (CHILDREN'S HOSPITAL MEDICAL CENTER), which analysed by microarray analysis the expression of hundreds of genes associated with the Arginine pathway in animals suffering from asthma, compared to control healthy animals. In this study, inventors have discovered a set of 291 signature genes that were consistently differently regulated in asthma models of disease. Moreover, several other genes have been identified in other studies as being asthma susceptibility genes, e.g. cytokines genes (IL13, IL4, IL10 and TNFα), cytokine receptor genes (IL14), HLA genes, and CD14 (OBER & HOFFJAN, Genes Immun., vol. 7, p:95-100, 2006).

However, although the familial aggregation of asthma has been explored and reported from multiple populations, relatively few strongly replicated asthma or allergy susceptibility genes have been identified to date, because these techniques have better utility for traditional Mendelian inherited disorders.

The second approach to discover gene in asthma susceptibility rely on the use of positional cloning in linkage and association studies within families as a mean of identifying markers that might be linked to allergies and asthma. Genome wide association studies rely on dense sets of SNPs across the genome to survey the most common genetic variants for a role in disease or to identify the heritable quantitative traits that are risk factors for diseases. The main strength of these studies is expected to lie in their ability to discover truly novel disease candidate genes, especially those associated with moderate risks. For this method to be statistically significative, thousands of cases and controls are needed and the study populations must be carefully characterized to allow the selection of patients who are likely to share a genetic cause of disease. Although genes associated with asthma or atopy have been found, only a small proportion of the susceptibility factors have so far been identified from regions of linkage (COOKSON & MOFFATT, N. Engl. J. Med., vol. 351, p:1794-6, 2004).

Because asthma can become progressively more severe over time, it is important to determine individuals that are susceptible to the disease at a young age. Thus, what is needed in the art is a mechanism for determining whether an individual is at risk for asthma. The present invention fulfils such a need.

### SUMMARY OF THE INVENTION

The present invention provides a method of testing a subject thought to have or be predisposed to having asthma, allergie, atopic disease or atopic sensitization which comprises the step of analyzing a biological sample from said subject for:
(i) detecting the presence of a mutation associated with the over-expression of the ORMDL3 gene, and/or
(ii) analyzing the expression of the ORMDL3 gene.

In another embodiment, the present invention provides also a use, for treating and/or preventing asthma, allergy, atopic disease or atopic sensitization in a subject, of a compound which specifically inhibits the expression of the ORMDL3 gene.

In still another embodiment, the present invention provides an in vitro method of selecting a compound, which can be useful for treating asthma, allergy, atopic disease or atopic sensitization, characterized in that said method comprises the steps of:
a) obtaining a cell expressing the ORMDL3 gene,
b) contacting said cell with at least one compound,
c) comparing the expression of the ORMDL3 gene in the cell between the steps a) and b), and
d) selecting the compound, which induces a lower level of expression of the ORMDL3 gene in the cell contacted to that compound.

### BRIEF DESCRIPTION OF THE DRAWINGS

FIG. 1a shows the distribution of total heritability for expression quantitative traits.

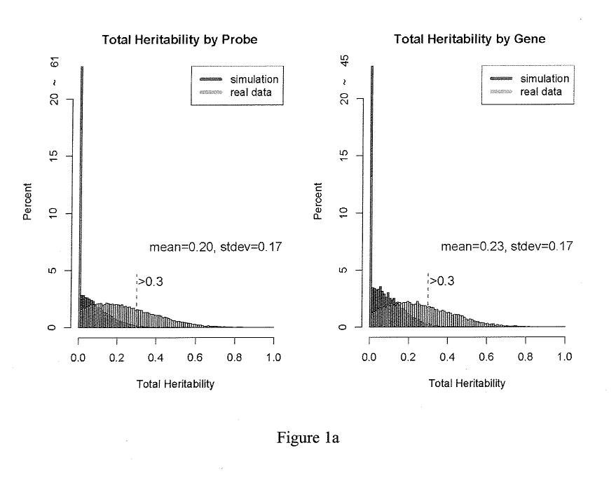

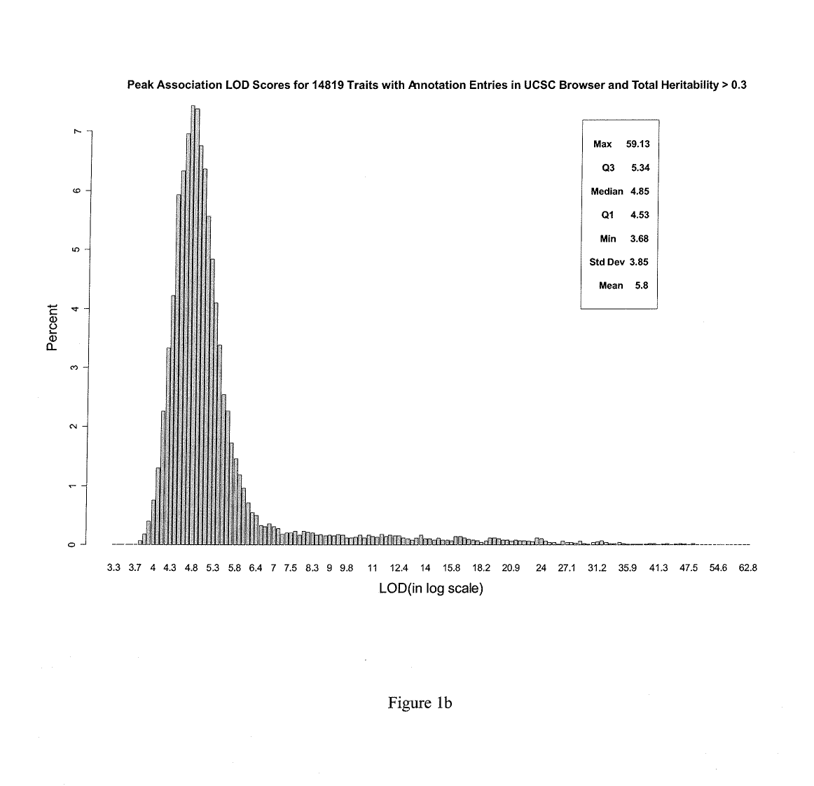

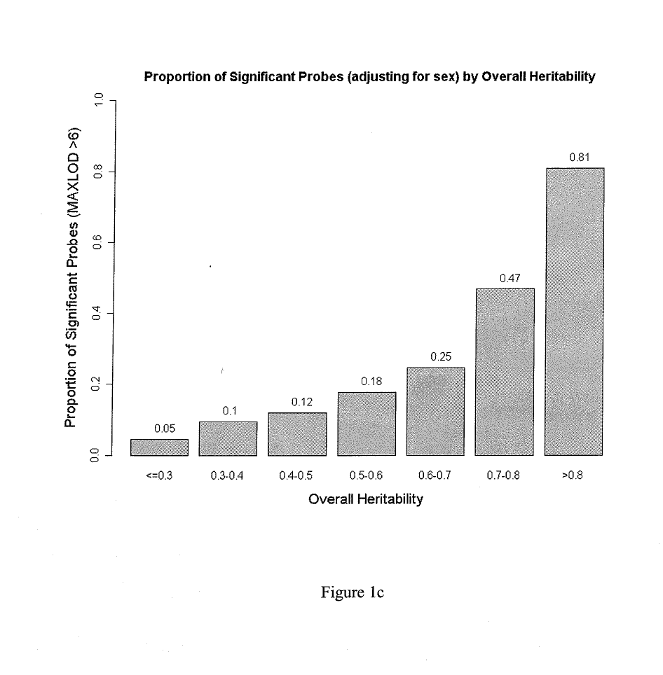

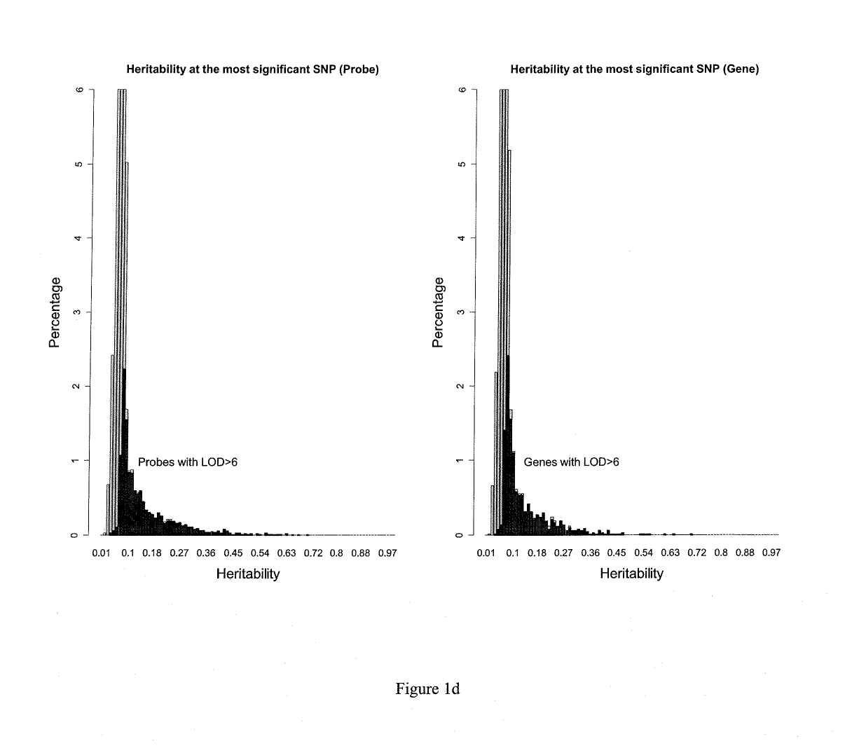

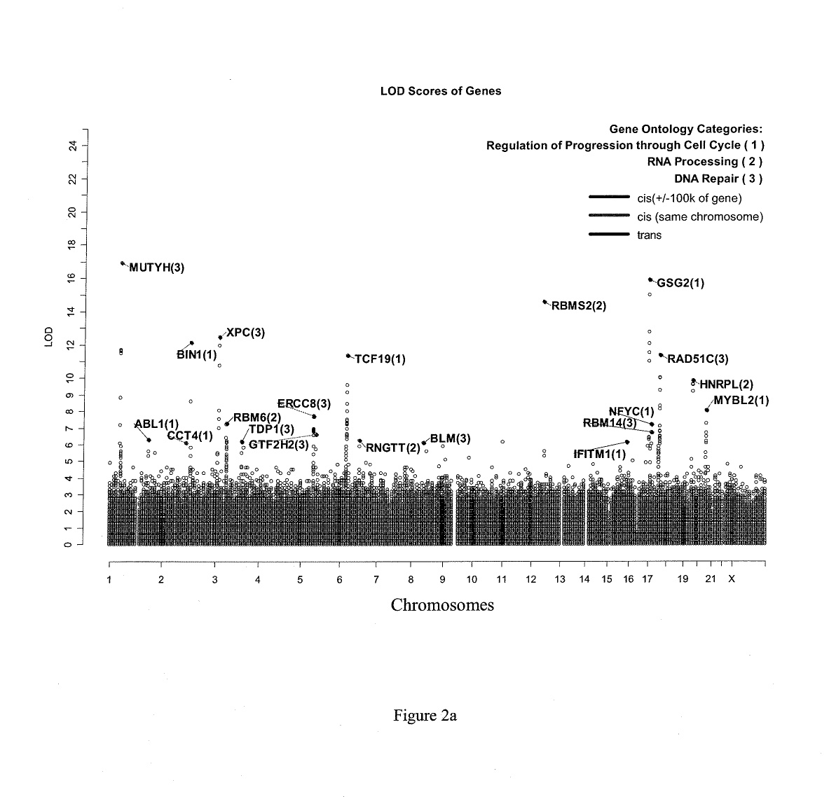

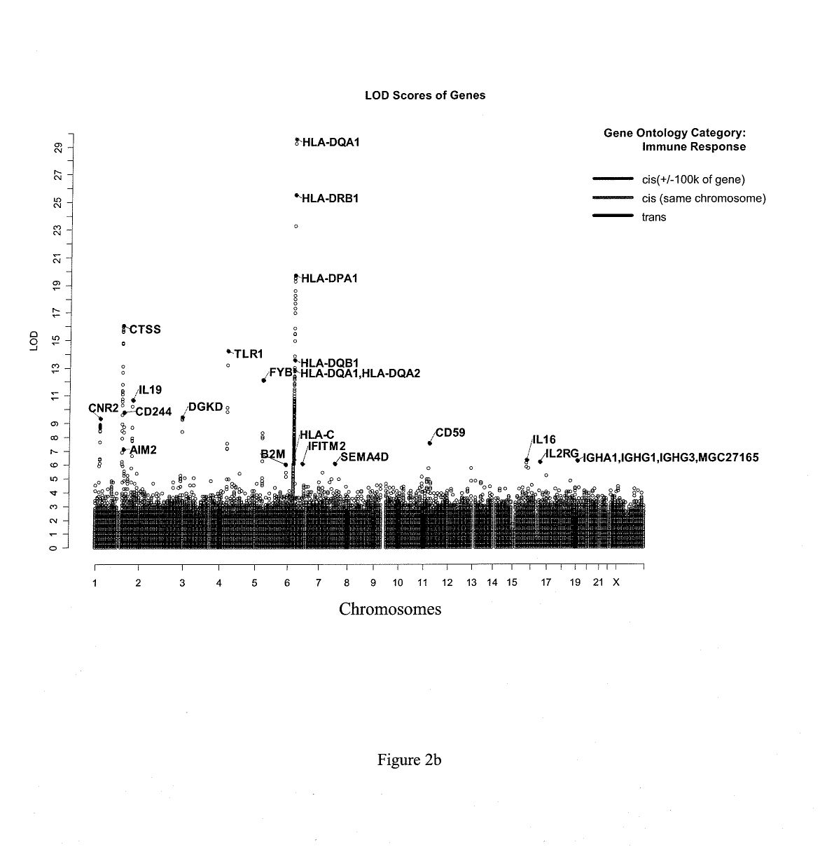

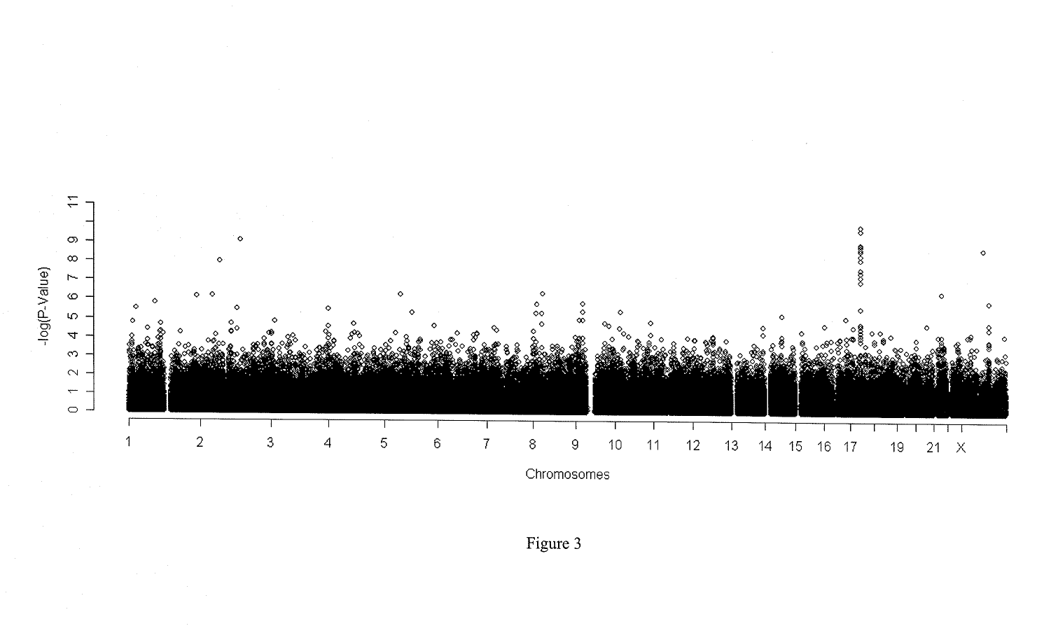

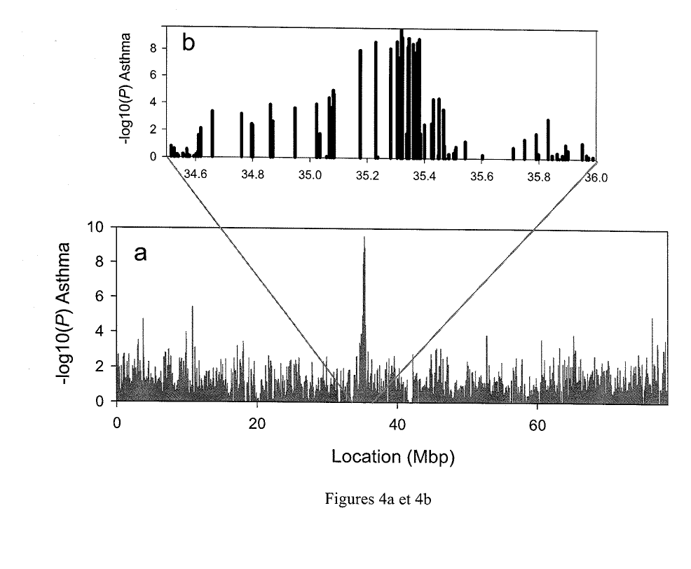

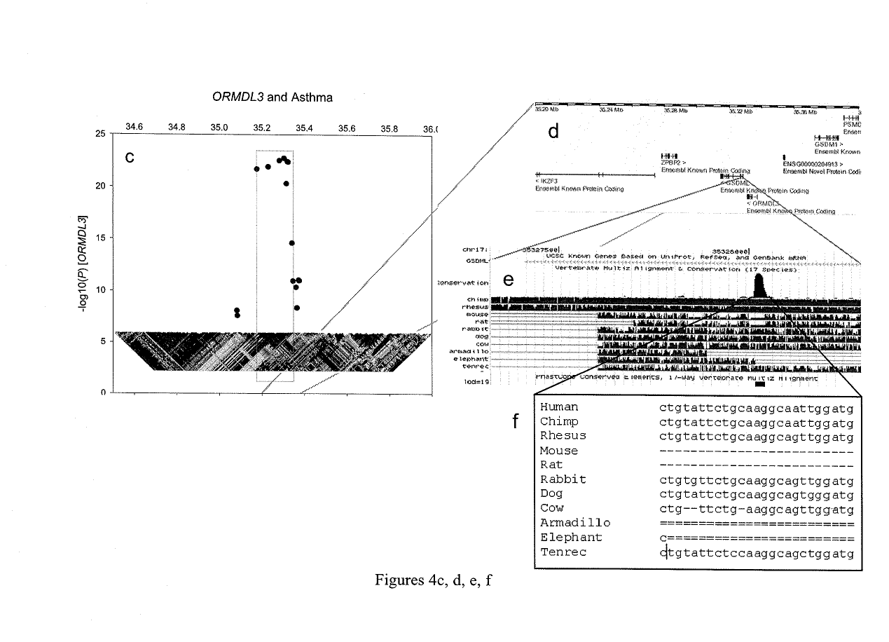

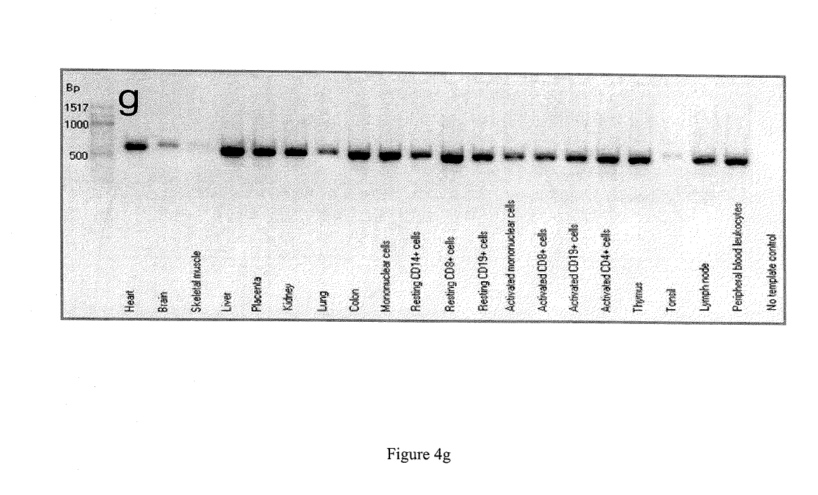

The FIG. 1b shows the distribution of LOD scores for association between 14819 traits and 408,273 SNP markers.

The FIG. 1c shows the proportion of traits with peak LOD >6 according to total trait heritability.

The FIG. 1d shows the total heritability accounted for by the SNP showing strongest association with gene expression levels.

The FIG. 2a shows the mapping of genes with GO-BP descriptors for cell cycle, DNA repair and RNA processing.

The FIG. 2b shows the mapping of 21 genes with GO-DP descriptors for immune responses.

The FIG. 3 shows the genome-wide association of 317,447 SNPs and asthma in 994 asthmatic children and 1,244 non-asthmatic children.

The FIG. 4 shows the association to asthma and transcript abundances of ORMDL3 on chromosome 17q21.

### DETAILED DESCRIPTION OF THE INVENTION

Despite advances in genotyping, the statistical power to identify genetic effects was constrained by sample size. Power may be enhanced by the study of intermediate phenotypes, in particular transcript abundances of genes (expression QTLs) that may be directly modified by polymorphism in regulatory elements (SCHADT et al., Nature, vol. 422, p: 297-302, 2003; MORLEY et al., Nature, vol. 430, p: 743-7, 2004).

Previous studies have shown the potential for eQTL mapping (SCHADT et al., 2003, abovementioned; MORLEY et al., 2004, abovementioned; CHEUNG et al., Nat. Genet., vol. 33, p: 422-5, 2003), but have examined limited numbers of transcripts or markers in a small number of CEPH (Centre d'Etude du Polymorphisme Humain) pedigrees. The EBV-transformed lymphoblastoid cell lines (EBVL) in these experiments had undergone multiple cell culture passages, which increases the risk of covert chromosomal rearrangements.

On the contrary, the present inventors have realized the first, and so far only, genome wide association study for asthma. In the initial phase of the study, more than 317000 SNPs were characterized in DNA from 994 patients with childhood onset asthma, and 1243 non-asthmatics, using family and case-control panels. Multiple markers on chromosome 17q21.1 were found to be strongly and reproducibly associated with the disease phenotype of interest. The association between the 17q21 locus and diagnosis of childhood asthma was independently replicated in 2320 subjects from a cohort of German children and in 3301 subjects from the British 1958 birth cohort (Moffat MF, Nature 2007, July 26;448(7152): 470-3). As the region of association on chromosome 17q21.1 spanned over 206 kb and included 19 annoted genes, the present inventors also identified that the SNPs associated with childhood asthma were consistently and strongly associated in cis with transcript levels of ORMDL3. Even if several known asthma candidate genes (STAT3, CRHR1, ITGB3, and TBX21) map to chromosome 17q21, this is the first time that ORMDL3 emerged as a candidate, as noticed by D. Vercelli in Nature Reviews Immunology 2008, vol 8, p 169-182. Since the discovery of the inventors, this result has been further confirmed by a Japanese genetic study showing that there is also a significant association between one SNP of ORMDL3 genotype and children with atopic asthma in 545 Japanese patients' vs. 738 healthy subjects (Hirota T, Journal of Allergy and Clinical Immunology, letters to the editor of march 2008, p 769-770).

Thus, the present invention is based on the discovery by the present inventors that an over-expression of the ORMDL3 transcripts and particular single nucleotide polymorphisms (SNP), which are associated with the over-expression of the ORMDL3 gene, are both correlated with asthma.

Thus, in a first aspect, the present invention provides a method of testing a subject thought to have or be predisposed to having asthma, allergy, atopic disease or atopic sensitization which comprises the step of analyzing a biological sample from said subject for:
(i) detecting the presence of a mutation associated with the over-expression of a gene of the ORM1-like gene (ORMDL) family, for example ORMDL3, and/or
(ii) analyzing the expression of a gene of the ORMDL family, for example ORMDL3.

Preferably, said method is useful for of testing a subject thought to have or be predisposed to having asthma.

Said method, by identifying an over-expression of a gene of the ORMDL family and/or by detecting the presence of a mutation associated with the over-expression of a gene of the ORM1-like gene (ORMDL) family in a subject enables to confirm that said subject has or is predisposed for having asthma.

As used herein, the term “subject” refers to a mammal, preferably a human.

As used herein, the expression “biological sample” refers to solid tissues such as, for example, a lung biopsy; buccal swab, fluids and excretions such as for example, sputum, induced sputum, blood, serum, plasma, urine. Preferably, said biological sample is a fluid sample and most preferably a blood sample.

As used herein, the expression “gene of the ORM1-like gene (ORMDL) family” refers to a conserved new family of endoplasmic reticulum membrane proteins, which has been described in HJELMQVIST et al. (Genome biology, vol. 3(6), RESEARCH0027, 2002). The ORMDL family includes the three ORMDL1, ORMDL2 and ORMDL3 genes in mammals. The sequences of the genes of the ORMDL family are well known by one of skill in the art and includes the gene ORMDL1 for Homo sapiens (mRNA: AF395704, protein: AAM43503), Mus musculus (mRNA: BCO23695, protein: AAH23695), Bos Taurus (mRNA: NM_{—}001014931, protein: NP_{—001014931}), Pongo pygmaeus (mRNA: CR859623, protein: Q5R8X5), and Macaca mulatta (mRNA: XM_{—001107686}, protein: XP_{—001107686}); the gene ORMDL2 for Homo sapiens (mRNA: NM_{—}014182, protein: NP_{—054901}), Mus musculus (mRNA: BC002146, protein: AAH02146), Bos Taurus (mRNA: BT021048, protein: AAX09065), and Pan troglodytes (mRNA: XM_{—}509124, protein: XP_{—}509124); and the gene ORMDL3 for Homo sapiens (mRNA: NM_{—}139280, SEQ ID NO: 1, protein: NP_{—}644809, SEQ ID NO:2, gene: corresponds to position 35330671 to 35331488 from NC_{—}000017.9), Mus musculus (mRNA: BC026412, protein: AAH26412), Pan troglodytes (mRNA: XM_{—}001171472, protein: XP_{—}001171472), Pongo pygmaeus (mRNA: CR860995, protein: Q5R570), and Rattus norvegicus (mRNA: AY539934, protein: Q6QI25).

Preferably, said gene of the ORM1-like gene (ORMDL) family is the ORMDL3 gene.

Typically, the mutation (s) associated with the over-expression of a gene of the ORM1-like gene (ORMDL) family corresponds to a single nucleotide polymorphism (SNP).

Preferably, said mutation(s) associated with the over-expression of a gene of the ORM1-like gene (ORMDL) family has the same chromosomal location as said gene. Consequently, said mutation can be located in the Open Reading Frame (ORF) of the ORM1-like (ORMDL) gene family or in the promoter region before the ORF of said gene. Preferably, said mutation is located on chromosome 17q21, in the ORF of the ORMDL3 gene or within an island of linkage disequilibrium between 35.2 and 35.4 Mbp on chromosome 17q21.

As an example and for a human subject, said mutation can be located on chromosome 2q32, on chromosome 12q13, or on chromosome 17q21, when said mutation is associated with an over-expression of the ORMDL1, ORMDL2 or ORMDL3 gene respectively.

Preferably, said mutation is a single nucleotide polymorphism associated with the over-expression of the ORMDL3 gene in a human, wherein said mutation is located on chromosome 17q21 and is selected from the group comprising rs9303277 (nucleotide N at position 128 of SEQ ID NO:3, wherein allele C is associated to asthma), rs11557467 (nucleotide N at position 128 of SEQ ID NO:4, wherein allele G is associated to asthma), rs8067378 (nucleotide N at position 128 of SEQ ID NO:5, wherein allele A is associated to asthma), rs2290400 (nucleotide N at position 128 of SEQ ID NO:6, wherein allele A is associated to asthma), rs7216389 (nucleotide N at position 128 of SEQ ID NO:7, wherein allele T is associated to asthma), rs4795405 (nucleotide N at position 128 of SEQ ID NO:8, wherein allele C is associated to asthma), rs8079416 (nucleotide N at position 128 of SEQ ID NO:9, wherein allele C is associated to asthma), rs4795408 (nucleotide N at position 128 of SEQ ID NO:10, wherein allele A is associated to asthma), rs3894194 (nucleotide N at position 128 of SEQ ID NO:11, wherein allele A is associated to asthma), rs3859192 (nucleotide N at position 128 of SEQ ID NO:12, wherein allele T is associated to asthma), rs9646419 (nucleotide N at position 128 of SEQ ID NO:13, wherein allele A is associated to asthma), rs14050 (nucleotide N at position 128 of SEQ ID NO:14, wherein allele C is associated to asthma), rs2941503 (nucleotide N at position 128 of SEQ ID NO:15, wherein allele A is associated to asthma), rs907087 (nucleotide N at position 128 of SEQ ID NO:16, wherein allele G is associated to asthma), rs2517954 (nucleotide N at position 128 of SEQ ID NO:17, wherein allele T is associated to asthma), rs1810132 (nucleotide N at position 128 of SEQ ID NO:18, wherein allele C is associated to asthma), rs907091 (nucleotide N at position 128 of SEQ ID NO:19, wherein allele T is associated to asthma), rs907092 (nucleotide N at position 128 of SEQ ID NO:20, wherein allele G is associated to asthma), rs10445308 (nucleotide N at position 128 of SEQ ID NO:21, wherein allele C is associated to asthma), rs10852936 (nucleotide N at position 128 of SEQ ID NO:22, wherein allele C is associated to asthma), rs1054609 (nucleotide N at position 128 of SEQ ID NO:23, wherein allele A is associated to asthma), rs8067378 (nucleotide N at position 128 of SEQ ID NO:24, wherein allele A is associated to asthma), rs2123685 (nucleotide N at position 128 of SEQ ID NO:25, wherein allele C is associated to asthma), rs8069176 (nucleotide N at position 128 of SEQ ID NO:26, wherein allele G is associated to asthma), rs2305480 (nucleotide N at position 128 of SEQ ID NO:27, wherein allele G is associated to asthma), rs2305479 (nucleotide N at position 128 of SEQ ID NO:14, wherein allele C is associated to asthma), rs11078926 (nucleotide N at position 128 of SEQ ID NO:29, wherein allele G is associated to asthma), rs1008723 (nucleotide N at position 128 of SEQ ID NO:30, wherein allele G is associated to asthma), rs4795400 (nucleotide N at position 128 of SEQ ID NO:31, wherein allele C is associated to asthma), rs7216389 (nucleotide N at position 128 of SEQ ID NO:32, wherein allele T is associated to asthma), rs9303281 (nucleotide N at position 128 of SEQ ID NO:33, wherein allele A is associated to asthma), rs7219923 (nucleotide N at position 128 of SEQ ID NO:34, wherein allele T is associated to asthma), rs3169572 (nucleotide N at position 128 of SEQ ID NO:35, wherein allele A is associated to asthma), rs4378650 (nucleotide N at position 128 of SEQ ID NO:36, wherein allele G is associated to asthma), rs8076131 (nucleotide N at position 128 of SEQ ID NO:37, wherein allele A is associated to asthma), rs3744246 (nucleotide N at position 128 of SEQ ID NO:38, wherein allele C is associated to asthma), rs4795402 (nucleotide N at position 128 of SEQ ID NO:39, wherein allele C is associated to asthma), rs4795403 (nucleotide N at position 128 of SEQ ID NO:40, wherein allele C is associated to asthma), rs4795404 (nucleotide N at position 128 of SEQ ID NO:41, wherein allele C is associated to asthma), rs4795405 (nucleotide N at position 128 of SEQ ID NO:42, wherein allele C is associated to asthma), rs4794820 (nucleotide N at position 128 of SEQ ID NO:43, wherein allele G is associated to asthma), rs7207600 (nucleotide N at position 128 of SEQ ID NO:44, wherein allele A is associated to asthma), rs6503525 (nucleotide N at position 128 of SEQ ID NO:45, wherein allele C is associated to asthma), rs8065126 (nucleotide N at position 128 of SEQ ID NO:46, wherein allele C is associated to asthma), rs3893044 (nucleotide N at position 128 of SEQ ID NO:47, wherein allele C is associated to asthma), rs4795408 (nucleotide N at position 128 of SEQ ID NO:48, wherein allele A is associated to asthma), rs7209742 (nucleotide N at position 128 of SEQ ID NO:49, wherein allele G is associated to asthma), rs8076474 (nucleotide N at position 128 of SEQ ID NO:50, wherein allele C is associated to asthma), rs1007654 (nucleotide N at position 128 of SEQ ID NO:51, wherein allele G is associated to asthma), rs1007655 (nucleotide N at position 128 of SEQ ID NO:52, wherein allele A is associated to asthma), rs2313640 (nucleotide N at position 128 of SEQ ID NO:53, wherein allele T is associated to asthma), rs7218742 (nucleotide N at position 128 of SEQ ID NO:54, wherein allele G is associated to asthma), rs7218321 (nucleotide N at position 128 of SEQ ID NO:55, wherein allele T is associated to asthma), rs7219080 (nucleotide N at position 128 of SEQ ID NO:56, wherein allele C is associated to asthma), rs6503526 (nucleotide N at position 128 of SEQ ID NO:57, wherein allele T is associated to asthma), rs6503527 (nucleotide N at position 128 of SEQ ID NO:58, wherein allele A is associated to asthma), rs3894194 (nucleotide N at position 128 of SEQ ID NO:59, wherein allele A is associated to asthma), rs7212938 (nucleotide N at position 128 of SEQ ID NO:60, wherein allele T is associated to asthma), rs2305479 (nucleotide N at position 128 of SEQ ID NO:61, wherein allele C is associated to asthma), rs2305480 (nucleotide N at position 128 of SEQ ID NO:62, wherein allele G is associated to asthma), rs2941503 (nucleotide N at position 128 of SEQ ID NO:63, wherein allele A is associated to asthma) and rs8076131 (nucleotide N at position 128 of SEQ ID NO:71 wherein allele C is associated to asthma).

Most preferably, said mutation is a single nucleotide polymorphism associated with the over-expression of the ORMDL3 gene in a human, wherein said mutation is located on chromosome 17q21 and is selected from the group comprising rs9303277 (nucleotide N at position 128 of SEQ ID NO:3, wherein allele C is associated to asthma), rs11557467 (nucleotide N at position 128 of SEQ ID NO:4, wherein allele G is associated to asthma), rs8067378 (nucleotide N at position 128 of SEQ ID NO:5, wherein allele A is associated to asthma), rs2290400 (nucleotide N at position 128 of SEQ ID NO:6, wherein allele A is associated to asthma), rs7216389 (nucleotide N at position 128 of SEQ ID NO:7, wherein allele T is associated to asthma), rs4795405 (nucleotide N at position 128 of SEQ ID NO:8, wherein allele C is associated to asthma), rs8079416 (nucleotide N at position 128 of SEQ ID NO:9, wherein allele C is associated to asthma), rs4795408 (nucleotide N at position 128 of SEQ ID NO:10, wherein allele A is associated to asthma), rs3894194 (nucleotide N at position 128 of SEQ ID NO:11, wherein allele T is associated to asthma), rs3859192 (nucleotide N at position 128 of SEQ ID NO:12, wherein allele T is associated to asthma) and rs8076131 (nucleotide N at position 128 of SEQ ID NO:71 wherein allele C is associated to asthma).

The skilled man will immediately appreciate that the information presented herein relating to the human ORMDL3 may easily be equated or correlated with a similar mutation at a corresponding location for the ORMDL3 gene from another species.

Typical techniques for detecting the mutation may include restriction fragment length polymorphism, hybridisation techniques, DNA sequencing, exonuclease resistance, microsequencing, solid phase extension using ddNTPs, extension in solution using ddNTPs, oligonucleotide ligation assays, methods for detecting single nucleotide polymorphisms such as dynamic allele-specific hybridisation, ligation chain reaction, mini-sequencing, DNA“chips”, allele-specific oligonucleotide hybridisation with single or dual-labelled probes merged with PCR or with molecular beacons, and others.

Preferably, said technique for detecting a mutation is selected in the group comprising methods for detecting single nucleotide polymorphisms.

Analyzing the expression of a gene of the ORMDL family, e.g. ORMDL3, may be assessed by any of a wide variety of well-known methods for detecting expression of a transcribed nucleic acid or translated protein.

In a preferred embodiment, the expression of a gene of the ORMDL family is assessed by analyzing the expression of mRNA transcript or mRNA precursors, such as nascent RNA, of said gene. Said analysis can be assessed by preparing mRNA/cDNA from cells in a biological sample from a subject, and hybridizing the mRNA/cDNA with a reference polynucleotide. The prepared mRNA/cDNA can be used in hybridization or amplification assays that include, but are not limited to, Southern or Northern analyses, polymerase chain reaction analyses, such as quantitative PCR (TaqMan), and probes arrays such as GeneChip™ DNA Arrays (AFFYMETRIX).

Advantageously, the analysis of the expression level of mRNA transcribed from a gene of the ORMDL family involves the process of nucleic acid amplification, e.g., by RT-PCR (the experimental embodiment set forth in U.S. Pat. No. 4,683,202), ligase chain reaction (BARANY, Proc. Natl. Acad. Sci. USA, vol. 88, p: 189-193, 1991), self sustained sequence replication (GUATELLI et al., Proc. Natl. Acad. Sci. USA, vol. 87, p: 1874-1878, 1990), transcriptional amplification system (KWOH et al., 1989, Proc. Natl. Acad. Sci. USA, vol. 86, p: 1173-1177, 1989), Q-Beta Replicase (LIZARDI et al., Biol. Technology, vol. 6, p: 1197, 1988), rolling circle replication (U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

In another preferred embodiment, the expression of a gene of the ORMDL family, e.g. ORMDL3, is assessed by analyzing the expression of the protein translated from said gene. Said analysis can be assessed using an antibody (e.g., a radio-labeled, chromophore-labeled, fluorophore-labeled, or enzyme-labeled antibody), an antibody derivative (e.g., an antibody conjugate with a substrate or with the protein or ligand of a protein of a protein/ligand pair (e.g., biotin-streptavidin)), or an antibody fragment (e.g., a single-chain antibody, an isolated antibody hypervariable domain, etc.) which binds specifically to the protein translated from a gene of the ORMDL family.

Said analysis can be assessed by a variety of techniques well known by one of skill in the art including, but not limited to, enzyme immunoassay (EIA), radioimmunoassay (RIA), Western blot analysis and enzyme linked immunoabsorbant assay (ELISA).

Polyclonal antibodies can be prepared by immunizing a suitable animal, such as mouse, rabbit or goat, with a protein encoded by a gene of the ORMDL family, e.g. ORMDL3, or a fragment thereof. The antibody titer in the immunized animal can be monitored over time by standard techniques, such as with an ELISA using immobilized polypeptide. At an appropriate time after immunization, e.g., when the specific antibody titers are highest, antibody producing cells can be obtained from the animal and used to prepare monoclonal antibodies (mAb) by standard techniques, such as the hybridoma technique originally described by KOHLER and MILSTEIN (Nature, vol. 256, p: 495-497, 1975), the human B cell hybridoma technique (KOZBOR et al., Immunol., vol. 4, p: 72, 1983), the EBV-hybridoma technique (COLE et al., In Monoclonal Antibodies and Cancer Therapy, Alan R. Liss, Inc., p: 77-96, 1985) or trioma techniques. The technology for producing hybridomas is well known (see generally Current Protocols in Immunology, COLIGAN et al. ed., John Wiley & Sons, New York, 1994). Hybridoma cells producing the desired monoclonal antibody are detected by screening the hybridoma culture supernatants for antibodies that bind the polypeptide of interest, e.g., using a standard ELISA.

The method of the invention may comprise comparing the level of expression of a gene of the ORMDL family, e.g. ORMDL3, in a biological sample from a subject with the normal expression level of said gene in a control. A significantly higher level of expression of said gene in the biological sample of a subject as compared to the normal expression level is an indication that the patient has or is predisposed to asthma.

An “over-expression” of a gene of the ORMDL family refers to an expression level in a biological sample that is greater than the standard error of the assay employed to assess expression, and is preferably at least 20% superior to the normal level of expression of said gene, preferably at least 50% superior to the normal level of expression of said gene, and most preferably at least 100% superior to the normal level of expression of said gene.

The “normal” level of expression of a gene of the ORMDL family is the level of expression of said gene in a biological sample of a subject not afflicted with asthma. Preferably, said normal level of expression is assessed in a control sample (e.g., sample from a healthy subject, which is not afflicted by asthma) and preferably, the average expression level of said gene in several control samples.

In another aspect, the present invention provides a use, for treating and/or preventing asthma, allergie, atopic disease or atopic sensitization in a subject, of a compound which specifically inhibits the expression of a gene of the ORMDL family, for example ORMDL3.

Preferably, the present invention provides a use, for treating and/or preventing asthma in a subject, of a compound which specifically inhibits the expression of a gene of the ORMDL family, e.g. ORMDL3.

Preferably, said compound specifically inhibiting the expression a gene of the ORMDL family is an oligonucleotide, which is selected from the group comprising anti-sense RNA and DNA molecules, ribozymes, siRNAs and aptamers.

More specifically, anti-sense RNA and DNA molecules and ribozymes function to inhibit the translation of ORMDL mRNA. Anti-sense RNA and DNA molecules act to directly block the translation of mRNA by binding to targeted mRNA and preventing protein translation. With regard to anti-sense DNA, oligodeoxyribonucleotides derived from the translation initiation site, between −10 and +10 regions of the ORMDL3 nucleotide sequence, are preferred. Ribozymes are enzymatic RNA molecules capable of catalyzing the specific cleavage of RNA. The mechanism of ribozyme action involves sequence specific hybridization of the ribozyme molecule to complementary target RNA, followed by a endonucleolytic cleavage. Within the scope of the invention are engineered hammerhead motif ribozyme molecules that specifically and efficiently catalyze endonucleolytic cleavage of ORMDL RNA sequences, preferably ORMDL3 RNA sequences. Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. Both anti-sense RNA and DNA molecules and ribozymes of the invention may be prepared by any method known in the art for the synthesis of RNA molecules. These include techniques for chemically synthesizing oligodeoxyribonucleotides well known in the art such as for example solid phase phosphoramidite chemical synthesis. Alternatively, RNA molecules may be generated by in vitro and in vivo transcription of DNA sequences encoding the anti-sense RNA molecule.

Such DNA sequences may be incorporated into a wide variety of vectors which incorporate suitable RNA polymerase promoters such as the T7 or SP6 polymerase promoters. Alternatively, anti-sense cDNA constructs that synthesize anti-sense RNA constitutively or inducibly, depending on the promoter used, can be introduced stably into cell lines.

Short interference RNA molecules (siRNA) can also be used for inhibiting the expression of a gene of the ORMDL family, e.g. ORMDL3. Said interference RNA molecules can be generated based on the genetic sequences of ORMDL genes, preferably ORMDL3. RNA interference (RNAi) is based on the degradation of particular target sequences by the design of short interference RNA oligo's (siRNA) which recognize the target sequence (here ORMDL) and subsequently trigger their degradation by a poorly understood pathway. In general siRNA duplexes are shorter than 30 nucleotides, because longer stretches of dsRNA activate the PKR pathway in mammalian cells which results in a global a-specific shut-down of protein synthesis. Preferably, the length of said siRNA is comprised between 15 and 25 bp (bases pair), and most preferably between 19 and 24 bp. The preparation and gene therapy vectors for the intracellular expression of siRNAs duplexes is disclosed in WO0244321. As an example of siRNA, one can cite SEQ ID NO:64 (CUAAGUACGACCAGAUCCA), SEQ ID NO: 65 (AAGGCAUGUGCUGCAACAC), SEQ ID NO: 66 (AGAAGAAGCCUCUGGACAC), SEQ ID NO: 67 (GUAGCCAACUUGGAGUAGC), SEQ ID NO: 68 (UCAAUAAGUACUGAGAGUG), SEQ ID NO: 69 (UAAGUACUGAGAGUGCAGC), and SEQ ID NO: 70 (AGUUCUUGACCAUCACACC).

RNA aptamers can also be used for inhibiting the expression of a gene of the ORMDL family. Said RNA aptamers can be generated against ORMDL genes, preferably ORMDL3. Recently, RNA aptamers have been used as therapeutic reagents for their ability to disrupt protein function. Selection of aptamers in vitro allows rapid isolation of extremely rare RNAs that have high specificity and affinity for specific proteins. Exemplary RNA aptamers are described in U.S. Pat. No. 5,270,163, in GOLD et al. (Nature, vol. 346, p: 818-822, 1990), and TUERK & GOLD (Science, vol. 249, p: 505-510, 1990). Unlike anti-sense compounds, whose targets are one dimensional lattices, RNA aptamers can bind to the three dimensional surfaces of a protein. Moreover, RNA aptamers can frequently discriminate finely among discrete functional sites of a protein (GOLD et al., Annu. Rev. Biochem., vol. 64, p: 763-797, 1995). As research and therapeutic reagents, aptamers not only have the combined advantages of antibodies and small molecular mass drugs, but in vivo production of RNA aptamers also can be controlled genetically. Such RNA expressing genes are usually smaller than protein-coding genes and can be inserted easily into gene therapy vectors.

Preferably, said oligonucleotide is a siRNA.

The oligonucleotide may be delivered in vivo alone or in association with a vector. In its broadest sense, a “vector” is any vehicle capable of facilitating the transfer of the siRNA to the cells and preferably cells over-expressing a gene of the ORMDL family, such as PBMC.

As an example, such oligonucleotide can be associated with non-lipid cationic polymers (WU and WU, J. Biol. Chem., vol. 263, p: 14621-4, 1988) or liposomes (BRIGHMAN et al., Am. J. Med. Sci., vol. 298, p: 278-81, 1989) to form complexes enhancing cellular uptake.

Advantageously, said compound specifically inhibiting the expression a gene of the ORMDL family may be associated with a pharmaceutically acceptable vehicle.

As an example of pharmaceutically acceptable vehicle, the composition may comprise emulsions, microemulsions, oil-in-water emulsions, anhydrous lipids and oil-in-water emulsions, other types of emulsions. The composition may also comprise one or more additives (e.g., diluents, excipients, stabilizers, preservatives). See, generally, Ullmann's Encyclopedia of Industrial Chemistry, 6^{th} Ed. (various editors, 1989-1998, Marcel Dekker); and Pharmaceutical Dosage Forms and Drug Delivery Systems (ANSEL et al., 1994, WILLIAMS & WILKINS).

In still another aspect, the present invention provides an in vitro method of selecting a compound, which can be useful for treating asthma, allergie, atopic disease or atopic sensitization, characterized in that said method comprises the steps of:
a) obtaining a cell expressing a gene of the ORMDL family;
b) contacting said cell with at least one compound,
c) comparing the expression of the gene of the ORMDL family in the cell between the steps a) and b),
d) selecting the compound, which induces a lower level of expression of the gene of the ORMDL family in the cell contacted to that compound.

Preferably, said method is useful for selecting a compound, which can be useful for treating asthma.

Preferably, said gene of the ORMDL family corresponds to ORMDL3.

Advantageously, said cell expressing a gene of the ORMDL family has been obtained from a subject suffering from asthma or has been derivated from a cell obtained from a subject suffering from asthma.

Preferably, said cell over-expressed the ORMDL3 gene, as compared to the corresponding cells from a subject which is not suffering from asthma.

Preferably, said cell corresponds to a peripheral blood leukocyte (PBL), and most preferably to a peripheral blood leukocyte immortalized with Epstein Barr Virus.

As used herein, the term “compound” refers to any type of molecules such as polypeptides, polynucleotides, sugars, lipids, or any other chemical compounds.

Methods for determining the expression of a gene of the ORMDL family are well known from one of skill in the art. As an example, one can cite the methods which have been described previously.

In the following, the invention is described in more detail with reference to amino acid sequences, nucleic acid sequences and the examples. Yet, no limitation of the invention is intended by the details of the examples. Rather, the invention pertains to any embodiment which comprises details which are not explicitly mentioned in the examples herein, but which the skilled person finds without undue effort.

### EXAMPLES

### 1) Subjects

We recruited a panel of 206 nuclear families (MRC-A) through a proband with severe (Step III) childhood asthma as part of the MRC UK National family collection. We included siblings regardless of asthma status⁶.

Children and their parents from the UK panels were administered a standard questionnaire (based on the American Thoracic Society and ISAAC questionnaires; Standards for the diagnosis and care of patients with chronic obstructive pulmonary disease (COPD) and asthma. This official statement of the American Thoracic Society was adopted by the ATS Board of Directors, November 1986. Am. Rev. Respir. Dis., vol. 136, p: 225-44, 1987) by a nurse practitioner or a doctor. Asthma was defined as a positive response to the question “Has your doctor ever told you that you have asthma”. Probands had Step 3 asthma or worse according to the BTS guidelines (high dose inhaled steroids, or low dose inhaled steroids and a long-acting β-agonist; British guideline on the management of asthma, Thorax, vol. 58 (Suppl 1), p:il-94, 2003).

All study methods were approved by the appropriate ethics committees.

### 2) EBVL Establishing

EBVL were established on probands and siblings. The transformation of the PBLs (Peripheral Blood Leukocytes) in all children in the panel was carried out by the ECACC (European Collection of Cell cultures). Previously transformed cryo-preserved EBV cell lines were grown as 500 ml roller cultures. Once log phase had been obtained, cells were pelleted, media discarded and a mixture of RLT buffer and β-mercaptoethanol added. Pellets were vortexed to ensure thorough re-suspension after which they were frozen at −70° C. and kept at −80° C. RNA was extracted in batches after cell homogenization using RNeasy® Maxi Kits (QIAGEN) and quality and quantity assessed.

### 3) Gene Expression

Global gene expression was measured.

10 μg of previously purified RNA was used to synthesize double-stranded cDNA using the One-cycle cDNA synthesis kit (AFFYMETRIX). Using the cDNA as a template, in vitro transcription of cRNA was carried out using the IVT kit (AFFYMETRIX) following the manufacturer's protocol. A hybridization cocktail was made according to protocol, using 15 μg of labelled, fragmented cRNA and hybridized to U133 Plus 2.0 GeneChips (AFFYMETRIX) for 16 hours at 45° C. in a rotating oven. GeneChips were washed and stained following the protocol and scanned on a high-resolution scanner (AGILENT TECHNOLOGIES).

All data from the gene expression experiment was normalized together using the RMA (Robust Multi-Array Average) package (IRIZARRY et al., Biostatistics, vol. 4, p: 249-64, 2003; BOLSTAD et al., Bioinformatics, vol. 19, p: 185-93, 2003) to remove any technical or spurious background variation. An inverse normalization transformation step was also applied to each trait to avoid any outliers. A variance components method was used to estimate heritability of each trait using the Merlin-regress (RandomSample option; ABECASIS et al., Nat. Genet., vol. 30, p: 97-101, 2002; SHAM et al., Am. J. Hum. Genet., vol. 71, p: 238-53, 2002).

Gene locations (to separate cis and trans-associations) were extracted from the UCSC table browser available at www.ncbi.nlm.nih.gov/RefSeq/. Annotations for individual transcripts on the Affymetrix arrays were extracted from the Affymetrix NetAffx database available at www.affymetrix.com.

FIG. 1a shows the distribution of total heritability for expression quantitative traits, wherein a reference null distribution was generated by permuting phenotypes across families. The left panel shows heritability estimates for each probe. In the right panel, expression measurements were averaged for all probes in the same gene before calculating heritability.

The results show that the H² for all the traits ranged between 0.0-1.0, with a mean of 0.203 and a 3^{rd} quartile (Q3) of 0.317 (FIG. 1a).

We focused subsequent analyses on 15,084 traits with H²>0.3.

### 4) Genotyping

Whole genome genotyping (WGGT) was carried out using Illumina Sentrix® Human-1 Genotyping BeadChip (GUNDERSON et al., Nat. Genet., vol. 37, p: 549-54, 2005) and Sentrix® HumanHap300 Genotyping BeadChips (STEEMERS et al., Nat. Methods., vol. 3, p: 31-3, 2006; ILLUMINA), according to manufacturers' instructions in a BeadLab with full automation.

All DNA samples were subjected to rigorous quality control to check for fragmentation and amplification. 20 μl of DNA at a concentration of 50 ng/μl was used for each array. DNA samples were tracked using a LIMS. The HumanHap300 Genotyping BeadChip was used with an Illumina LIMS while the Sentrix Human-1 Genotyping BeadChip was tracked through the ILLUMINA process by hand. Groups of 24 samples were batched. Five percent of the samples were selected from different batches, re-genotyped and the results compared to the original data. No sample discrepancies were detected. Raw data was analyzed using GTS Image and extracted for statistical analysis.

378 offspring were successfully genotyped with the Illumina Sentrix® HumanHap300 BeadChip (derived from HapMap Phase I), producing 317,497 SNPs with 34.7% average heterozygosity and 119,372,631 genotypes (99.5% call rate). 830 offspring and parents were genotyped using the Illumina Sentrix® Human-1 Genotyping BeadChip (concentrated on genes and surrounding sequences), producing an additional 91,293 SNPs with 36.0% average heterozygosity and 89,815,992 genotypes (99.0% call rate).

Tests of Hardy-Weinberg equilibrium were performed in cases and controls using the genhwi procedure (available at http://www.biostat-resources.com/stata/) and Stata™ version 9.2, and SNPs showing Hardy-Weinberg disequilibrium in controls (χ²>25) were excluded. As the data comprised a mixture of unrelated and related cases and controls, we used logistic regression models with robust sandwich estimation of the variance (WILLIAMS, Biometrics, vol. 56, p: 645-6, 2000) as implemented in the Stata™ logit function to model clustering of sibling's genotypes. Simulations using the MRC-A family structures (data available on request) confirmed that this method appropriately controls the type I error. Heterogeneity of association between the two main strata (UK and Germany) was assessed by a weighted linear combination test using the results of an additive-effects-only regression analysis within each stratum. X-linked markers were analysed by fitting an additive-effects-only logit model which equates the risks of male hemizygotes with female homozygotes. The TRANSMIT program (CLAYTON, Am. J. Hum. Genet., vol. 65, p: 1170-7, 1999) was used to analyse nuclear family data (including parental genotypes) using the sandwich variance estimation option to robustly incorporate information from multiple affected siblings. The overall statistical significance of the asthma GWA results was assessed by application of the QVALUE (available at http://faculty.washington.edu/˜jstorey/qvalue/) software package (STOREY & TIBSHIRANI, Proc. Natl. Acad. Sci. USA, vol. 100, p: 9440-5, 2003).

The test for Hardy-Weinberg equilibrium in 392 unrelated individuals identified 2.7% and 2.1% of SNPs deviating at the 0.01 level in the 100K and 300K panels respectively. We found only 0.412 Mendelian errors per SNP amongst the 102018 markers typed on all family members. These SNPs were included in analyses.

The results have shown that the Minor Allele Frequency (MAF) was predominately >0.1 (Supplementary FIG. 1). Our 408,273 successfully genotyped SNPs contained 372,821 SNPs from the 2,236,212 common SNPs (MAF >0.05) in HapMap database. The total coverage of HapMap at R²>0.8 was 1,794,828 SNPs (80.3%), and for R²>0.5 was 2,043,847 SNPs (91.4%). For the 317,149 SNPs tested in the 300K panel the coverage for R²>0.8 was 1,762,736 SNPs (78.8%) and for R²>0.5 was 2,032,597 SNPs (90.9%).

### 5) Genetic Association to eQTL

We applied the method of genomic control (DEVLIN et al., Theor. Popul. Biol., vol. 60, p: 155-66, 2001) and derived a coefficient of 1.0099, indicating negligible population stratification.

Association analysis was applied with Merlin (FASTASSOC option; ABECASIS et al., 2002, abovementioned). We estimated an additive effect for each SNP and tested its significance using a score test that adjusts for familiality and including sex in the model, which takes into account uncertainty in the inference of missing genotypes. In the absence of a positive genomic control test we did not adjust for stratification. We probabilistically inferred missing genotypes and adjusted for familiality, but not for linkage signal. A significance level of 0.05 and a Bonferroni correction applied to each expression trait individually led to a P-value threshold of 1.2×10⁻⁷, corresponding to a lod score of 6.076. This equates to a false discovery rate of 0.049 for 408,273 SNPs tested for association with 15,084 transcripts (BENJAMINI & HOCHBERG, J. R. Statist. Soc. B., vol. 57, p: 289-300, 1995).

The FIG. 1b shows the distribution of peak lod scores for association between traits with annotation entries in the UCSC browser and H²>0.3, and SNP markers.

We found that the 14,819 traits with annotation entries in the UCSC browser and H²>0.3 had a minimum lod score for association of 3.683, and a maximum of 59.128 (median 4.853, Q3 5.339) (FIG. 1b). We estimated the threshold for genome wide significance to be a peak lod score >6.076. We found the false discovery rate (FDR) for a lod score of 5.5 to be 0.152, for a lod of 6 to be 0.056, for a lod of 7.0 to be 0.0067, and for a lod of 8 to be 0.0008.

When the peak associated lod was >6, 78% of the most strongly associated SNPs were on the same chromosome as the transcripts and 89% of these SNPs were within the transcript. Only 22% of transcripts were associated with SNPs on different chromosomes. The median distance between a transcript and SNPs associated with its expression was approximately 9.2 kb.

The FIG. 1c shows the proportion of traits with peak LOD >6 according to total trait heritability, wherein the results show that traits with higher total heritability were, on average, more mappable.

On average, we observed a higher proportion of peak LOD scores above our threshold (lod >6.0) for traits with higher H². For example, 81% of traits with H²>0.80 showed association to a SNP in our panels with lod >6.0 (FIG. 1c). This proportion decreased to only 5% for traits with a heritability of <30%. This may suggest that increases in sample size or selection for trait distribution may have more impact on the power of GWA studies than increases in marker density.

Five of our 50 most significant eQTL had been identified previously (RPS26, IRF5, LRAP, CHI3L2 and HSD17B12; MORLEY et al., 2004, abovementioned), showing eQTL mapping to be reproducible. The presence of numerous other highly significant eQTLs in our study is attributable to the larger sample size and more extensive exploration of the genome that we have undertaken, and creates a resource for the systematic mapping of complex disease genes.

For each trait, we examined the SNP showing strongest association.

The FIG. 1d shows the total heritability accounted for by the SNP showing strongest association with gene expression levels. On average, we observed larger effects when the peak associated SNP mapped in cis (red bars, within 100 kb of target) of the probe or gene being evaluated than when it mapped in trans (blue bars). The right panel shows results after averaging all probes for each gene, as in FIG. 1a. The % scale on the y axis is truncated at 6.0.

The mean H² attributable to this SNP was 0.066 (SD 0.029, max 0.707, sum 3629) compared to 0.203 for the overall H² of these traits (SD 0.170, max 1.0, sum 11104). This indicated that genotypes at a single SNP could explain 32.5% of the heritable component of these traits (FIG. 1d). The H² attributable to a single SNP rose to 0.151 for the traits with a peak lod >6.0, accounting for 29.3% of the H² for these traits.

We explored the unexplained heritability by testing for dominance and interaction effects amongst the 5824 genes with H²>0.3 that could not be mapped (maxlod <6) under the additive model. We identified 408 genes under a dominant model with P<6.12×10⁻⁸ (Bonferroni correction for 2*408273 tests), which was less than the 634 genes observed in analysis with randomized genotypes. We tested for interactions amongst the top 100 SNPs for each of the 5824 genes, and found 363 genes had P<6×10⁻⁸ (Bonferroni correction for 2*408273+10000 tests), compared to 210 genes observed in analysis with randomized genotypes. The results suggest that dominance had a minimal effect on gene transcription, and that interactions were minor.

Using a threshold R²>0.8 in our genotype data we found that 79036 SNPs in the gene-targeted 100K panel were tagged by the HapMap derived 300K panel and 75098 SNPs in 300K panel could be tagged by the 100K panel. With both panels combined, we mapped 592 genes in cis with lod for association >6. 209 genes mapped to the same SNP, 216 genes mapped at different cis SNPs in each panel, 64 genes mapped only using the 100K panel and 103 genes were mapped only using the 300K panel. Thus 10.8% of hits were specific to the 100K panel, 17.4% of hits were specific to the 300K and 71.8% of hits were identified with both panels. Consequently we typed only the 300K panel in the case-referent samples.

### 6) Gene Ontology

We used Gene Ontology analyses to identify genes that were significantly enriched amongst highly heritable traits.

Transcripts and genes were matched to Gene Ontology (ASHBURNER et al., Nat. Genet., vol. 25, p: 25-9, 2000)categories using the HG-U133 Plus 2.0 annotation file (Dec. 19, 2005) available from AFFYMETRIX NetAffx (available at http://www.affymetrix.com/support/technical/byproduct.affx?product=hg-u133-plus). We excluded GO annotations inferred from electronic annotation only, as these are considered less reliable (HARRIS et al., Nucleic Acids Res., vol. 32, p: D258-61, 2004). We investigated the average heritability of the transcripts assigned to each GO category using the statistic

$${Z}_{{H}_{i}^{2}} = \frac{{H}_{i}^{2} - μ}{σ / \sqrt{{n}_{i}}}$$

where Hᵢ² is the average heritability of the transcripts assigned to the i^{th} GO category, nᵢ is the number of transcripts in the i^{th} category, μ and σ are the overall mean and standard deviation of all heritability estimates, respectively.

The results are presented in the table 1 as follows:

TABLE 1
Gene Ontology of exceptionally heritable traits.

| | GO | | Z for |
|---|---|---|---|
| GO-Biological Process | ID | H² | H² |
| response to unfolded protein | 6986 | 0.38 | 9.03 |
| regulation of progression through cell cycle | 74 | 0.26 | 8.20 |
| RNA processing | 6396 | 0.30 | 7.85 |
| DNA repair | 6281 | 0.29 | 7.81 |
| protein folding | 6457 | 0.30 | 7.80 |
| immune response | 6955 | 0.26 | 7.62 |
| regulation of I-kappaB kinase/NF-kappaB cascade | 43123 | 0.28 | 6.84 |
| mitosis | 7067 | 0.30 | 5.82 |
| intracellular signaling cascade | 7242 | 0.26 | 5.72 |
| regulation of transcription | 45449 | 0.29 | 5.70 |
| regulation of viral genome replication | 45069 | 0.39 | 5.47 |
| protein biosynthesis | 6412 | 0.26 | 5.45 |
| vesicle-mediated transport | 16192 | 0.27 | 5.02 |
| cytokinesis | 910 | 0.32 | 4.94 |
| protein complex assembly | 6461 | 0.25 | 4.67 |
| DNA replication | 6260 | 0.27 | 4.61 |
| phosphoinositide-mediated signaling | 48015 | 0.32 | 4.59 |
| humoral immune response | 6959 | 0.30 | 4.47 |
| apoptosis | 6915 | 0.24 | 4.42 |

The analysis compared the mean total H² of transcripts in an individual GO category (GOID) with the mean total H² of all 54675 transcripts.

The results show that the most highly heritable GO biological process was “response to unfolded proteins”. This group contained numerous chaperonins and heat shock proteins (CRNN, 7 DNAJ family members, HERPUD1, 16 HSPA, B, C or D family members, SERPINH1, TOR1A and 1B, TRA1 and TXNDC4).

The individual variation in response to unfolded proteins may represent an evolutionary response to cellular stress, and these genes could be candidates in the study of neurodegenerative diseases and aging processes.

The FIG. 2a shows the mapping of genes with GO-BP descriptors for cell cycle, DNA repair and RNA processing, and overlays the results of genome wide association analysis for 20 genes annotated in the cell cycle, DNA repair and RNA processing GO Biological Process categories. The peak SNP for each trait is annotated with the gene name if the strength of association exceeds LOD >6.0.

The results show that the genes regulating progression through cell cycle, RNA processing, and DNA repair were also exceptionally heritable (FIG. 2a).

The evolutionary advantage of individual variation in these genes is unclear. These genes may be relevant candidates for the investigation of inherited susceptibility to cancer.

It has been shown previously that genes expressed in EBVL are enriched in GO categories of immune response (MONKS et al., Am. J. Hum. Genet., vol. 75, p: 1094-105, 2004).

The mapping of 21 genes with GO-DP descriptors for immune responses is shown in FIG. 2b.

The results show the significant heritability that we observed to these traits (FIG. 2b), which emphasizes their importance to inflammatory diseases. Several of these genes have an effect on asthma, including those of the MHC, CHIA (acid chitinase; ZHU et al., Science, vol. 304, p: 1678-82, 2004; BIERBAUM et al., Am. J. Respir. Crit. Care. Med., vol. 172, p: 1505-9, 2005) and IL16 (DE BIE et al., Clin Exp Allergy, vol. 32, p: 1651-8, 2002; BURKART et al., J. Allergy. Clin. Immunol., vol117, p: 86-91, 2006). IL2RG is a key signalling component of many interleukin receptors, and CD59 is ubiquitously expressed and protects cells against activated complement.

### 7) Asthma

In order to test for association with the categorical phenotype of asthma, we further genotyped 728 physician-diagnosed asthmatic and 694 non-asthmatic children recruited from Germany and Austria, together with 437 non-asthmatic Caucasian UK controls.

Asthma cases from the Multicentre Asthma Genetics in Childhood Study (MAGICS) were diagnosed by a paediatric pulmonologist or allergologist based on clinical examination, case history and objective tests of lung function. Asthmatics (mean age 10.95 years) were recruited from 7 centres located in Germany and Austria (Wesel, Bochum Cologne, Freiberg, Munich, Feldkirch and Vienna), and as a reference, 800 German children (mean age 9.62 years) from Dresden (n=400) and Munich (n=400) were randomly drawn from all German children with DNA available in the cross sectional International Study of Asthma and Allergy in Childhood, phase II (WEILAND et al., Eur. Respir. J., vol. 24, p: 406-12, 2004).

All study methods were approved by the appropriate ethics committees.

We used the Illumina Sentrix® HumanHap300 BeadChip as described previously, producing 317,447 SNPs and 452,361,975 genotypes (99.5% call rate).

We tested for association to asthma in the combined data set of 994 asthmatics and 1,224 non-asthmatics.

The FIG. 3 shows the genome-wide association of 317,447 SNPs and asthma in 994 asthmatic children and 1,244 non-asthmatic children.

The results show that numerous markers on chromosome 17q21 show association to asthma in the region of maximum association (FIG. 3). We calculated that a 1% FDR corresponded to a P≦6.8×10⁻⁷, and found 20 SNPs below this threshold. The 5% FDR threshold was 5.0×10⁻⁶ (34 SNPs) and the 10% threshold was 1.1×10⁻⁵ (37 SNPs), suggesting the presence of multiple susceptibility alleles for asthma.

Analysis for population stratification in the combined dataset found a genomic control parameter of 1.079, reflecting differences in allele frequencies within the European population. We controlled for admixture by modeling population stratification ¹³ with 102 randomly selected informative markers in HWE in a backward stepwise regression for the 34 SNPs showing association to asthma below the 5% FDR threshold. This had a minimal effect on association to any of these loci.

Potential associations below the 1% FDR included several markers that had only been typed in one of the panels or had exceptionally low minor allele frequencies. When these were excluded 16 SNPs remained andare presented in the following table 2:

TABLE 2
Markers that exceed the FDR 5% threshold for association with
childhood asthma after adjustment for population stratification

| | | Location | Uncorrected | Corrected |
|---|---|---|---|---|
| Marker | Chromosome | (pb) | P-value | P-value |
| rs10924993 | 1 | 238,526,408 | 7.E−07 | 3.E−06 |
| rs6716266 | 2 | 69,098,732 | 1.E−08 | 9.E−08 |
| rs8179521 | 2 | 127,867,394 | 3.E−06 | 8.E−07 |
| rs3791244 | 2 | 138,448,235 | 7.E−10 | 5.E−08 |
| rs4512342 | 8 | 32,727,416 | 4.E−07 | 2.E−09 |
| rs2666781 | 10 | 22,245,682 | 4.E−06 | 4.E−06 |
| rs907092 | 17 | 35,175,785 | 1.E−09 | 8.E−07 |
| rs9303277 | 17 | 35,229,995 | 2.E−09 | 5.E−08 |
| rs11557467 | 17 | 35,282,160 | 8.E−10 | 3.E−08 |
| rs8067378 | 17 | 35,304,874 | 9.E−10 | 4.E−08 |
| rs2305480 | 17 | 35,315,722 | 3.E−09 | 5.E−07 |
| rs2290400 | 17 | 35,319,766 | 2.E−10 | 4.E−09 |
| rs7216389 | 17 | 35,323,475 | 9.E−11 | 3.E−09 |
| rs4795405 | 17 | 35,341,943 | 2.E−09 | 5.E−07 |
| rs2037986 | 21 | 28,398,348 | 4.E−07 | 6.E−07 |
| rs2311978 | X | 75,705,247 | 2.E−09 | 2.E−07 |

Strikingly, 7 of the 12 markers still below the 1% FDR threshold mapped to a 112 kb interval on chromosome 17q21.

Several other markers in this interval also showed strong evidence of association. The table 3a shows the results of association analysis for markers on chromosome 17q21 in the UK, German and combined panels used in the GWA study. Markers are either from the HumanHap300 set (origin=GWA) or supplementary markers of the region selected from dsSNP.

TABLE 3a

-Log10 (p-value)

| Marker | Location | UK | Germany | Combined |
|---|---|---|---|---|
| rs907092 | 35,175,785 | 4.97 | 4.66 | 8.92 |
| rs9911688 | 35,197,327 | 0.31 | 0.49 | 0.63 |
| rs9303277 | 35,229,995 | 4.40 | 5.02 | 8.79 |
| rs2060941 | 35,236,409 | 0.47 | 0.00 | 0.27 |
| rs9908694 | 35,251,298 | 0.28 | 0.64 | 0.76 |
| rs11557467 | 35,282,160 | 4.57 | 5.15 | 9.07 |
| rs8067378 | 35,304,874 | 4.34 | 5.31 | 9.03 |
| rs2123685 | 35,307,415 | 0.95 | 5.67 | 4.76 |
| rs8069176 | 35,310,723 | 3.61 | 6.42 | 9.42 |
| rs2305480 | 35,315,722 | 3.80 | 5.24 | 8.48 |
| rs2305479 | 35,315,743 | 5.07 | 6.62 | 11.05 |
| rs2290400 | 35,319,766 | 3.96 | 9.81 | 6.44 |
| rs4795400 | 35,320,546 | 3.56 | 4.60 | 7.60 |
| rs7216389 | 35,323,475 | 4.25 | 6.39 | 10.04 |
| rs9303281 | 35,327,572 | 4.59 | 7.81 | 11.74 |
| rs7219923 | 35,328,044 | 5.04 | 7.39 | 11.78 |
| rs3169572 | 35,330,938 | 0.52 | 0.84 | 1.12 |
| rs4378650 | 35,334,391 | 4.43 | 7.11 | 10.90 |
| rs8076131 | 35,334,438 | 3.80 | 6.53 | 9.72 |
| rs3744246 | 35,337,876 | 0.88 | 1.40 | 1.97 |
| rs4795402 | 35,338,911 | 1.52 | 2.10 | 3.22 |
| rs4795403 | 35,339,248 | 1.29 | 1.46 | 2.40 |
| rs4795404 | 35,339,317 | 1.13 | 1.27 | 2.07 |
| rs4795405 | 35,341,943 | 4.06 | 5.26 | 8.70 |
| rs4794820 | 35,342,870 | 3.90 | 5.56 | 8.87 |
| rs7207600 | 35,345,186 | 2.16 | 3.72 | 5.38 |
| rs8079416 | 35,346,239 | 2.42 | 6.49 | 8.23 |
| rs6503525 | 35,348,700 | 2.87 | 7.01 | 9.26 |
| rs8065126 | 35,352,561 | 2.36 | 4.40 | 6.23 |
| rs3893044 | 35,356,542 | 3.28 | 4.77 | 7.47 |
| rs4795408 | 35,361,153 | 2.11 | 6.08 | 7.53 |
| rs7209742 | 35,362,234 | 2.52 | 4.38 | 6.36 |
| rs8076474 | 35,364,760 | 2.52 | 4.45 | 6.43 |
| rs1007654 | 35,364,880 | 2.08 | 4.07 | 5.65 |
| rs1007655 | 35,364,945 | 2.40 | 4.01 | 5.90 |
| rs2313640 | 35,365,371 | 2.50 | 4.12 | 6.09 |
| rs7218742 | 35,367,887 | 2.59 | 4.16 | 6.22 |
| rs7218321 | 35,367,995 | 2.33 | 3.84 | 5.66 |
| rs7219080 | 35,368,042 | 1.79 | 4.35 | 5.60 |
| rs6503526 | 35,368,124 | 2.93 | 6.67 | 8.98 |
| rs6503527 | 35,368,245 | 2.43 | 3.83 | 5.75 |
| rs3902025 | 35,372,780 | 2.67 | 4.92 | 7.06 |
| rs3894194 | 35,375,519 | 1.92 | 6.63 | 7.72 |
| rs7212938 | 35,376,206 | 2.05 | 0.79 | 1.05 |
| rs3859192 | 35,382,174 | 1.98 | 7.30 | 6.00 |
| rs921651 | 35,387,448 | 0.25 | 1.27 | 1.25 |
| rs8066582 | 35,400,455 | 1.36 | 1.38 | 2.35 |
| rs8080546 | 35,400,492 | 0.27 | 1.59 | 1.52 |
| rs2071369 | 35,425,831 | 0.96 | 0.90 | 1.55 |
| rs2302776 | 35,431,675 | 1.60 | 3.04 | 4.19 |
| rs3934886 | 35,449,021 | 0.04 | 0.68 | 0.44 |
| rs11078936 | 35,451,440 | 1.92 | 2.91 | 4.38 |
| rs868150 | 35,466,885 | 1.53 | 1.65 | 2.78 |
| rs7502966 | 35,470,048 | 0.92 | 0.59 | 1.19 |
| rs1568400 | 35,474,634 | 0.25 | 0.04 | 0.10 |
| rs939348 | 35,485,379 | 0.59 | 0.28 | 0.64 |
| rs3744805 | 35,501,880 | 0.39 | 0.77 | 0.95 |
| rs2071427 | 35,508,018 | 0.30 | 0.17 | 0.34 |
| rs2269457 | 35,508,215 | 0.24 | 0.65 | 0.72 |
| rs2071570 | 35,510,616 | 0.63 | 0.23 | 0.61 |
| rs7211770 | 35,542,529 | 0.27 | 1.02 | 1.05 |
| rs2280400 | 35,602,653 | 0.05 | 0.55 | 0.33 |
| rs13706 | 35,710,677 | 0.15 | 1.27 | 0.65 |

The results identified significant SNP markers having a p value equal or greater than 6. The results further established that the patterns of association for the chromosome 17q21 markers were similar in both the UK family panel and the German case-referent panel. There was no evidence of heterogeneity of the association or of significant allele frequency differences in cases or controls from the UK and Germany.

We selected 27 markers from the dbSNP database that were within or adjacent to the strongly associated interval for genotyping. These exhibited similar patterns and strength association as the GWA markers (Table 3a).

Table 3b shows results of family based analysis (TRANSMIT) in 116 SNPs from the association region on chromosome 17q21 genotyped in parents and children of UK childhood asthma family.

TABLE 3b

| Marker | Location (bp) | -Log10(pvalue) |
|---|---|---|
| rs1989955 | 34,515,900 | 0.96 |
| rs16230 | 34,521,940 | 0.42 |
| rs498973 | 34,541,591 | 0.50 |
| rs16495 | 34,541,657 | 0.43 |
| rs645167 | 34,553,967 | 0.01 |
| rs563525 | 34,562,881 | 0.19 |
| rs16522 | 34,570,514 | 0.41 |
| rs544198 | 34,572,555 | 0.14 |
| rs657672 | 34,572,591 | 0.56 |
| rs657723 | 34,572,629 | 0.15 |
| rs16525 | 34,575,131 | 0.27 |
| rs3744353 | 34,587,769 | 078 |
| rs2240083 | 34,604,929 | 0.21 |
| rs521633 | 34,608,592 | 0.23 |
| rs16530 | 34,610,560 | 0.32 |
| rs11657409 | 34,621,682 | 0.22 |
| rs2338755 | 34,672,843 | 2.21 |
| rs8079590 | 34,679,727 | 2.12 |
| rs588193 | 34,693,965 | 2.12 |
| rs2302073 | 34,710,868 | 2.30 |
| rs2338800 | 34,751,675 | 2.12 |
| rs6503513 | 34,815,139 | 1.77 |
| rs9646419 | 34,850,711 | 2.54 |
| rs4795369 | 34,862,646 | 1.97 |
| rs11655550 | 34,863,649 | 3.12 |
| rs4390625 | 34,873,873 | 1.76 |
| rs12948906 | 34,898,380 | 1.89 |
| rs6503518 | 34,913,995 | 2.07 |
| rs7503705 | 34,923,230 | 2.08 |
| rs1619021 | 34,992,800 | 2.06 |
| rs9889354 | 35,011,493 | 0.05 |
| rs907094 | 35,043,897 | 0.83 |
| rs3764352 | 35,044,465 | 0.84 |
| rs881844 | 35,063,744 | 1.25 |
| rs1877031 | 35,067,606 | 1.19 |
| rs876493 | 35,078,071 | 1.10 |
| rs14050 | 35,081,598 | 2.52 |
| rs2941503 | 35,082,271 | 2.67 |
| rs907087 | 35,082,313 | 2.52 |
| rs2517954 | 35,097,076 | 2.55 |
| rs2517955 | 35,097,207 | 2.00 |
| rs2517956 | 35,097,385 | 2.45 |
| rs1810132 | 35,119,531 | 2.61 |
| rs907091 | 35,175,268 | 5.30 |
| rs907092 | 35,175,785 | 3.84 |
| rs10445308 | 35,191,573 | 3.96 |
| rs9911634 | 35,197,257 | 1.10 |
| rs10852936 | 35,285,240 | 3.62 |
| rs1054609 | 35,286,803 | 3.48 |
| rs8067378 | 35,304,874 | 5.51 |
| rs2123685 | 35,307,415 | 1.25 |
| rs8069176 | 35,310,723 | 3.98 |
| rs2305480 | 35,315,722 | 3.75 |
| rs2305479 | 35,315,743 | 5.32 |
| rs11078926 | 35,316,502 | 3.36 |
| rs1008723 | 35,319,793 | 4.65 |
| rs4795400 | 35,320,546 | 3.20 |
| rs7216389 | 35,323,475 | 5.27 |
| rs9303281 | 35,327,572 | 4.73 |
| rs7219923 | 35,328,044 | 5.01 |
| rs3169572 | 35,330,938 | 1.11 |
| rs4378650 | 35,334,391 | 4.67 |
| rs8076131 | 35,334,438 | 3.62 |
| rs3744246 | 35,337,876 | 1.58 |
| rs4795402 | 35,338,911 | 2.24 |
| rs4795403 | 35,339,248 | 1.47 |
| rs4795404 | 35,339,317 | 1.47 |
| rs4795405 | 35,341,943 | 3.30 |
| rs4794820 | 35,342,870 | 3.35 |
| rs7207600 | 35,345,186 | 1.56 |
| rs6503525 | 35,348,700 | 3.79 |
| rs8065126 | 35,352,561 | 2.03 |
| rs3893044 3 | 5,356,542 | 2.12 |
| rs4795408 | 35,361,153 | 3.33 |
| rs7209742 | 35,362,234 | 2.04 |
| rs8076474 | 35,364,760 | 1.90 |
| rs1007654 | 35,364,880 | 1.73 |
| rs1007655 | 35,364,945 | 1.73 |
| rs2313640 | 35,365,371 | 1.90 |
| rs7218742 | 35,367,887 | 1.73 |
| rs7218321 | 35,367,995 | 1.47 |
| rs7219080 | 35,368,042 | 1.28 |
| rs6503526 | 35,368,124 | 3.87 |
| rs6503527 | 35,368,245 | 1.73 |
| rs3902025 | 35,372,780 | 1.76 |
| rs3894194 | 35,375,519 | 3.76 |
| rs7212938 | 35,376,206 | 3.11 |
| rs8077456 | 35,382,291 | 1.49 |
| rs3859188 | 35,396,504 | 0.00 |
| rs9915252 | 35,398,614 | 1.11 |
| rs2012 | 35,407,610 | 0.13 |
| rs2227319 | 35,424,371 | 0.77 |
| rs25645 | 35,426,669 | 1.23 |
| rs1042658 | 35,427,428 | 0.88 |
| rs1045929 | 35,428,952 | 1.04 |
| rs12309 | 35,428,988 | 1.45 |
| rs709591 | 35,429,087 | 1.00 |
| rs3213762 | 35,432,153 | 0.79 |
| rs2302775 | 35,436,370 | 0.17 |
| rs3935280 | 35,442,581 | 0.80 |
| rs868150 | 35,466,885 | 0.74 |
| rs7502966 | 35,470,048 | 0.18 |
| rs1879265 | 35,484,902 | 0.09 |
| rs3744805 | 35,501,880 | 0.20 |
| rs939347 | 35,510,219 | 0.49 |
| rs3744806 | 35,605,152 | 0.26 |
| rs4566234 | 35,667,875 | 0.00 |
| rs9916460 | 35,681,591 | 0.07 |
| rs9908257 | 35,681,648 | 0.16 |
| rs2077464 | 35,700,090 | 0.05 |
| rs13706 | 35,710,677 | 0.09 |
| rs2120200 | 35,713,901 | 0.09 |

SNPs from the region that we had typed in whole families in the MRC-A panel showed significant associations in a family based test, with SNP markers having a p value equal or greater than 2.5.

A supplemental analysis reveals an association with asthma of ORMDL3 gene (chromosome 17q21), which is the third member of a novel class of genes of unknown function that encode transmembrane proteins anchored in the endoplasmic reticulum (ER; HJELMQVIST et al., 2002, abovementioned).

The FIG. 4 shows the association to asthma and increased transcript abundances of ORMDL3 on chromosome 17q21. a) Mapping of association to asthma on chromosome 17, b) Detail of association to SNPs on chromosome 17q21, c) association to ORMDL3 transcript abundance with the same markers. A GOLD plot 30 of linkage disequilibrium (LD) between markers is also shown, with red indicating high LD and blue low. The central island of LD, which contains maximum association to ORMDL3 and asthma, is contained within the grey rectangle, d) genes contained within the associated interval e) homology plot from region of maximum association f) sequence homology from intron I of GSDML, g) RT-PCR (34 cycles) of ORMDL3 cDNA from representative tissues (Clontech Ltd).

We examined multiple tissue cDNA panels by RT-PCR, and found that ORMDL3 to be expressed in many tissues, particularly liver and peripheral blood lymphocytes (FIG. 4g).

The table 4a shows the association of asthma and ORMDL3 to chromosome 17q21.1 in combined panels.

TABLE 4a

| | | -log10(P) | | | | |
|---|---|---|---|---|---|---|
| | | | | | freq. | freq. |
| | Location | [ORMDL3] | | | allele 1 | allele 2 |
| Marker | (Mbp) | Family | allele_1 | allele_2 | asthmatic | asthmatic |
| rs9303277 | 35.230 | 21.9 | C | T | 0.58 | 0.42 |
| rs11557467 | 35.282 | 22.5 | G | T | 0.58 | 0.42 |
| rs8067378 | 35.305 | 22.7 | A | G | 0.58 | 0.42 |
| rs2290400 | 35.320 | 22.4 | A | G | 0.59 | 0.41 |
| rs7216389 | 35.323 | 22.4 | T | C | 0.58 | 0.42 |
| rs4795405 | 35.342 | 14.6 | C | T | 0.63 | 0.37 |
| rs8079416 | 35.346 | 10.9 | C | T | 0.53 | 0.47 |
| rs4795408 | 35.361 | 10.3 | A | G | 0.52 | 0.48 |
| rs3894194 | 35.376 | 11.0 | A | C | 0.53 | 0.47 |
| rs3859192 | 35.382 | 3.5 | T | C | 0.51 | 0.49 |

SNPs are from the Illumina 300K panel. The results are calculated with logistic regression models. Association to ORMDL3 transcript abundance is shown for comparison and is based on the family panel only.

TABLE 4b
Transmission disequilibrium tests of association
of asthma to chromosome 17q21.1 in family panels.
TRANSMIT

| | Location | Informative | | | | | | -log10(P) |
|---|---|---|---|---|---|---|---|---|
| Marker | (Mbp) | Transmissions | χ² | -log10(P) | GRR | 95% | CI | [ORMDL3] |
| rs907091 | 35.175 | 245 | 20.8 | 5.3 | 1.82 | 1.41 | 2.40 | 24.1 |
| rs907092 | 35.175 | 232 | 14.4 | 3.8 | 1.66 | 1.28 | 2.20 | 21.7 |
| rs10445308 | 35.191 | 275 | 15.0 | 4.0 | 1.61 | 1.27 | 2.07 | 21.4 |
| rs2305480 | 35.315 | 246 | 14.0 | 3.7 | 1.63 | 1.27 | 2.13 | 20.3 |
| rs1008723 | 35.320 | 245 | 18.0 | 4.6 | 1.74 | 1.35 | 2.29 | 23.7 |
| rs7216389 | 35.323 | 225 | 20.7 | 5.3 | 1.87 | 1.44 | 2.50 | 22.4 |
| rs9303281 | 35.327 | 246 | 18.2 | 4.7 | 1.75 | 1.36 | 2.29 | 25.0 |
| rs7219923 | 35.330 | 250 | 17.2 | 4.5 | 1.71 | 1.33 | 2.23 | 25.2 |
| rs3894194 | 35.375 | 246 | 14.1 | 3.8 | 1.63 | 1.27 | 2.13 | 8.5 |

SNPs are from the Illumina 100K panel. Results of association to asthma are calculated using TRANSMIT. Association to ORMDL3 transcript abundance is shown for comparison, and was calculated using MERLIN.

The results establish that 13 of the SNPs below the 5% FDR mapped to a 206 kb interval on chromosome 17 (Table 4a, FIG. 4 and FIG. 5), with the most strongly associated SNP was rs7216389 (P=9.1×10⁻¹¹). In fact, the T allele at rs7216389 has a frequency 62% amongst asthmatics compared to 52% in non-asthmatics. Association to chromosome 17 markers was present in the family and the case-referent panel, and was highly significant in both case/control and family-based tests of association (Table 4b).

A forward stepwise regression with all markers in the 34.5 to 36 Mb interval on chromosome 17 identified 3 SNPs (rs7216389, rs11650680 and rs3859192,) showing independent statistical effects on asthma (global P=7.1×10⁻¹³). This finding was consistent with the presence of more than one functional SNP, or the presence of untyped functional SNPs in incomplete LD with the typed markers.

We then examined the locus with our eQTL data, and found that transcripts in the ORMDL3 gene were strongly (P<10⁻²²) and consistently positively associated to the same SNPs as asthma (FIG. 4) (correlation between asthma and ORMDL3 P values=0.67, P=0.004). No other transcripts from the region showed significant relationship to the disease-associated markers, indicating that the same polymorphisms that are associated with ORMDL3 transcript levels are also associated with asthma susceptibility.

Finally, the SNPs showing the strongest association to asthma and ORMDL3 transcript abundances are contained within an island of linkage disequilibrium between 35.2 and 35.4 Mbp on chromosome 17q21 (FIGS. 4b and 4c).

### 8) Transcription Factor Binding Analyses by Electrophoretic Mobility Shift Assay

To investigate putative changes in transcription factor binding depending on the genotype of the polymorphism on position rs8076131 which has a high linkage disequilibrium, Electrophoretic Mobility Shift Assays (EMSA) were performed.

Briefly, oligonucleotides (33 bp) labelled with ³²P, carrying either the wild-type or the polymorphic allele were incubated with nuclear extract from the Jurkat T-cell line. The binding of the transcription factor AP1 (Nakabeppu Y et al, Cell 1988, December 2; 55(5):907-15) was then investigated by gel electrophoresis on a polyacrylamide gel. Two competitors of AP1 consensus sites (100-fold molar excess) were used for each experiment.

As the speed at which the different molecules move through the gel was changed in the lane containing the polymorphic allele, we were able to determinate that AP1 protein was bound to the transcription binding site in the polymorphic allele and not in the wild-type allele (data not shown).

1. A method of testing a subject thought to have or be predisposed to having asthma, allergy, atopic disease or atopic sensitization, which comprises the step of analyzing a biological sample from said subject for:
(i) detecting the presence of a mutation associated with the over-expression of the ORMDL3 gene, and/or
(ii) analyzing the expression of the ORMDL3 gene.

2. The method according to claim 1, wherein said subject is thought to have or be predisposed to having asthma.

3. The method according to claim 1, wherein said subject is a mammal, preferably a human.

4. The method according to claim 1, wherein said mutation(s) associated with the over-expression of the ORMDL3 gene corresponds to a single nucleotide polymorphism (SNP).

5. The method according to claim 1, wherein said mutation(s) associated with the over-expression of the ORMDL3 gene is located on chromosome 17q21.

6. The method according to claim 1, wherein said mutation is selected from the group consisting of rs9303277 (nucleotide N at position 128 of SEQ ID NO:3, wherein allele C is associated to asthma), rs11557467 (nucleotide N at position 128 of SEQ ID NO:4, wherein allele G is associated to asthma), rs8067378 (nucleotide N at position 128 of SEQ ID NO:5, wherein allele A is associated to asthma), rs2290400 (nucleotide N at position 128 of SEQ ID NO:6, wherein allele A is associated to asthma), rs7216389 (nucleotide N at position 128 of SEQ ID NO:7, wherein allele T is associated to asthma), rs4795405 (nucleotide N at position 128 of SEQ ID NO:8, wherein allele C is associated to asthma), rs8079416 (nucleotide N at position 128 of SEQ ID NO:9, wherein allele C is associated to asthma), rs4795408 (nucleotide N at position 128 of SEQ ID NO:10, wherein allele A is associated to asthma), rs3894194 (nucleotide N at position of SEQ ID NO:11, wherein allele T is associated to asthma), rs3859192 (nucleotide N at position 128 of SEQ ID NO:12, wherein allele T is associated to asthma), rs9646419 (nucleotide N at position 128 of SEQ ID NO:13, wherein allele A is associated to asthma), rs14050 (nucleotide N at position 128 of SEQ ID NO:14, wherein allele C is associated to asthma), rs2941503 (nucleotide N at position 128 of SEQ ID NO:15, wherein allele A is associated to asthma), rs907087 (nucleotide N at position 128 of SEQ ID NO:16, wherein allele G is associated to asthma), rs2517954 (nucleotide N at position 128 of SEQ ID NO:17, wherein allele T is associated to asthma), rs1810132 (nucleotide N at position 128 of SEQ ID NO:18, wherein allele C is associated to asthma), rs907091 (nucleotide N at position 128 of SEQ ID NO:19, wherein allele T is associated to asthma), rs907092 (nucleotide N at position 128 of SEQ ID NO:20, wherein allele G is associated to asthma), rs10445308 (nucleotide N at position 128 of SEQ ID NO:21, wherein allele C is associated to asthma), rs10852936 (nucleotide N at position 128 of SEQ ID NO:22, wherein allele C is associated to asthma), rs1054609 (nucleotide N at position 128 of SEQ ID NO:23, wherein allele A is associated to asthma), rs8067378 (nucleotide N at position 128 of SEQ ID NO:24, wherein allele A is associated to asthma), rs2123685 (nucleotide N at position 128 of SEQ ID NO:25, wherein allele C is associated to asthma), rs8069176 (nucleotide N at position 128 of SEQ ID NO:26, wherein allele G is associated to asthma), rs2305480 (nucleotide N at position 128 of SEQ ID NO:27, wherein allele G is associated to asthma), rs2305479 (nucleotide N at position 128 of SEQ ID NO:14, wherein allele C is associated to asthma), rs11078926 (nucleotide N at position 128 of SEQ ID NO:29, wherein allele G is associated to asthma), rs1008723 (nucleotide N at position 128 of SEQ ID NO:30, wherein allele G is associated to asthma), rs4795400 (nucleotide N at position 128 of SEQ ID NO:31, wherein allele C is associated to asthma), rs7216389 (nucleotide N at position 128 of SEQ ID NO:32, wherein allele T is associated to asthma), rs9303281 (nucleotide N at position 128 of SEQ ID NO:33, wherein allele A is associated to asthma), rs7219923 (nucleotide N at position 128 of SEQ ID NO:34, wherein allele T is associated to asthma), rs3169572 (nucleotide N at position 128 of SEQ ID NO:35, wherein allele A is associated to asthma), rs4378650 (nucleotide N at position 128 of SEQ ID NO:36, wherein allele G is associated to asthma), rs8076131 (nucleotide N at position 128 of SEQ ID NO:37, wherein allele A is associated to asthma), rs3744246 (nucleotide N at position 128 of SEQ ID NO:38, wherein allele C is associated to asthma), rs4795402 (nucleotide N at position 128 of SEQ ID NO:39, wherein allele C is associated to asthma), rs4795403 (nucleotide N at position 128 of SEQ ID NO:40, wherein allele C is associated to asthma), rs4795404 (nucleotide N at position 128 of SEQ ID NO:41, wherein allele C is associated to asthma), rs4795405 (nucleotide N at position 128 of SEQ ID NO:42, wherein allele C is associated to asthma), rs4794820 (nucleotide N at position 128 of SEQ ID NO:43, wherein allele G is associated to asthma), rs7207600 (nucleotide N at position 128 of SEQ ID NO:44, wherein allele A is associated to asthma), rs6503525 (nucleotide N at position 128 of SEQ ID NO:45, wherein allele C is associated to asthma), rs8065126 (nucleotide N at position 128 of SEQ ID NO:46, wherein allele C is associated to asthma), rs3893044 (nucleotide N at position 128 of SEQ ID NO:47, wherein allele C is associated to asthma), rs4795408 (nucleotide N at position 128 of SEQ ID NO:48, wherein allele A is associated to asthma), rs7209742 (nucleotide N at position 128 of SEQ ID NO:49, wherein allele G is associated to asthma), rs8076474 (nucleotide N at position 128 of SEQ ID NO:50, wherein allele C is associated to asthma), rs1007654 (nucleotide N at position 128 of SEQ ID NO:51, wherein allele G is associated to asthma), rs1007655 (nucleotide Nat position 128 of SEQ ID NO:52, wherein allele A is associated to asthma), rs2313640 (nucleotide N at position 128 of SEQ ID NO:53, wherein allele T is associated to asthma), rs7218742 (nucleotide N at position 128 of SEQ ID NO:54, wherein allele G is associated to asthma), rs7218321 (nucleotide N at position 128 of SEQ ID NO:55, wherein allele T is associated to asthma), rs7219080 (nucleotide N at position 128 of SEQ ID NO:56, wherein allele C is associated to asthma), rs6503526 (nucleotide N at position 128 of SEQ ID NO:57, wherein allele T is associated to asthma), rs6503527 (nucleotide N at position 128 of SEQ ID NO:58, wherein allele A is associated to asthma), rs3894194 (nucleotide N at position 128 of SEQ ID NO:59, wherein allele A is associated to asthma), rs7212938 (nucleotide N at position 128 of SEQ ID NO:60, wherein allele T is associated to asthma), rs2305479 (nucleotide N at position 128 of SEQ ID NO:61, wherein allele C is associated to asthma), rs2305480 (nucleotide N at position 128 of SEQ ID NO:62, wherein allele G is associated to asthma), rs2941503 (nucleotide N at position 128 of SEQ ID NO:63, wherein allele A is associated to asthma), and rs8076131 (nucleotide N at position 128 of SEQ ID NO:71 wherein allele C is associated to asthma).

7. The method according to claim 1, wherein the step (ii) of analyzing the expression of the ORMDL3 gene is assessed by detecting expression of a transcribed nucleic acid or translated protein.

8. The method according to claim 7, wherein said method comprises the step of comparing the level of expression of the ORMDL3 gene in said biological sample with the normal expression level of said gene in a control.

9. A method for treating and/or preventing asthma, allergy, atopic disease or atopic sensitization, comprising administering to a subject in need thereof a compound which specifically inhibits the expression of the ORMDL3 gene.

10. The method according to claim 9, wherein said method is for treating and/or preventing asthma in a subject.

11. The method according to claim 10, wherein said compound specifically inhibiting the expression of the ORMDL3 gene is an oligonucleotide, which is selected from the group consisting of anti-sense RNA and DNA molecules, ribozymes, siRNAs and aptamers.

12. The method according to claim 10, wherein said oligonucleotide is a siRNA, which is selected from the group consisting of

| | |
|---|---|
| SEQ ID NO: 64 | (CUAAGUACGACCAGAUCCA), |
| SEQ ID NO: 65 | (AAGGCAUGUGCUGCAACAC), |
| SEQ ID NO: 66 | (AGAAGAAGCCUCUGGACAC), |
| SEQ ID NO: 67 | (GUAGCCAACUUGGAGUAGC), |
| SEQ ID NO: 68 | (UCAAUAAGUACUGAGAGUG), |
| SEQ ID NO: 69 | (UAAGUACUGAGAGUGCAGC), |
| and | |
| SEQ ID NO: 70 | (AGUUCUUGACCA MAC ACC). |

13. An in vitro method of selecting a compound, which can be useful for treating asthma, allergy, atopic disease or atopic sensitization, wherein said method comprises the steps of:
a) obtaining a cell expressing the ORMDL3 gene,
b) contacting said cell with at least one compound,
c) comparing the expression of the ORMDL3 gene in the cell between the steps a) and b), and
d) selecting the compound, which induces a lower level of expression of the ORMDL3 gene in the cell contacted to that compound.

14. The method according to claim 13, wherein said method is for selecting a compound, which can be useful for treating asthma.
